SEQUENCE LISTING (7 sequences)

SEQ ID NO: 1 - protein, 887 aa, Bifidobacterium bifidum
VEDATRSDSTTQMSSTPEVVYSSAVDSKQNRTSDFDANWKFMLSDSVQAQDPAFDDSAWQQVDLPHDYSITQKYSQSNEAESAYLPGGTGWYRKSFTIDRDLAGKRIAINFDGVYMNATVWFNGVKLGTHPYGYSPFSFDLTGNAKFGGENTIVVKVENRLPSSRWYSGSGIYRDVTLTVTDGVHVGNNGVAIKTPSLATQNGGDVTMNLTTKVANDTEAAANITLKQTVFPKGGKTDAAIGTVTTASKSIAAGASADVTSTITAASPKLWSIKNPNLYTVRTEVLNGGKVLDTYDTEYGFRWTGFDATSGFSLNGEKVKLKGVSMHHDQGSLGAVANRRAIERQVEILQKMGVNSIRTTHNPAAKALIDVCNEKGVLVVEEVFDMWNRSKNGNTEDYGKWFGQAIAGDNAVLGGDKDETWAKFDLTSTINRDRNAPSVIMWSLGNEMMEGISGSVSGFPATSAKLVAWTKAADSTRPMTYGDNKIKANWNESNTMGDNLTANGGVVGTNYSDGANYDKIRTTHPSWAIYGSETASAINSRGIYNRTTGGAQSSDKQLTSYDNSAVGWGAVASSAWYDVVQRDFVAGTYVWTGFDYLGEPTPWNGTGSGAVGSWPSPKNSYFGIVDTAGFPKDTYYFYQSQWNDDVHTLHILPAWNENVVAKGSGNNVPVVVYTDAAKVKLYFTPKGSTEKRLIGEKSFTKKTTAAGYTYQVYEGSDKDSTAHKNMYLTWNVPWAEGTISAEAYDENNRLIPEGSTEGNASVTTTGKAAKLKADADRKTITADGKDLSYIEVDVTDANGHIVPDAANRVTFDVKGAGKLVGVDNGSSPDHDSYQADNRKAFSGKVLAIVQSTKEAGEITVTAKADGLQSSTVKIATTAVPGTSTEKT

SEQ ID NO: 2 - protein, 965 aa, Bifidobacterium bifidum
VEDATRSDSTTQMSSTPEVVYSSAVDSKQNRTSDFDANWKFMLSDSVQAQDPAFDDSAWQQVDLPHDYSITQKYSQSNEAESAYLPGGTGWYRKSFTIDRDLAGKRIAINFDGVYMNATVWFNGVKLGTHPYGYSPFSFDLTGNAKFGGENTIVVKVENRLPSSRWYSGSGIYRDVTLTVTDGVHVGNNGVAIKTPSLATQNGGDVTMNLTTKVANDTEAAANITLKQTVFPKGGKTDAAIGTVTTASKSIAAGASADVTSTITAASPKLWSIKNPNLYTVRTEVLNGGKVLDTYDTEYGFRWTGFDATSGFSLNGEKVKLKGVSMHHDQGSLGAVANRRAIERQVEILQKMGVNSIRTTHNPAAKALIDVCNEKGVLVVEEVFDMWNRSKNGNTEDYGKWFGQAIAGDNAVLGGDKDETWAKFDLTSTINRDRNAPSVIMWSLGNEMMEGISGSVSGFPATSAKLVAWTKAADSTRPMTYGDNKIKANWNESNTMGDNLTANGGVVGTNYSDGANYDKIRTTHPSWAIYGSETASAINSRGIYNRTTGGAQSSDKQLTSYDNSAVGWGAVASSAWYDVVQRDFVAGTYVWTGFDYLGEPTPWNGTGSGAVGSWPSPKNSYFGIVDTAGFPKDTYYFYQSQWNDDVHTLHILPAWNENVVAKGSGNNVPVVVYTDAAKVKLYFTPKGSTEKRLIGEKSFTKKTTAAGYTYQVYEGSDKDSTAHKNMYLTWNVPWAEGTISAEAYDENNRLIPEGSTEGNASVTTTGKAAKLKADADRKTITADGKDLSYIEVDVTDANGHIVPDAANRVTFDVKGAGKLVGVDNGSSPDHDSYQADNRKAFSGKVLAIVQSTKEAGEITVTAKADGLQSSTVKIATTAVPGTSTEKTVRSFYYSRNYYVKTGNKPILPSDVEVRYSDGTSDRQNVTWDAVSDDQIAKAGSFSVAGTVAGQKISVRVTMIDEIGAL

SEQ ID NO: 3 - protein, 1038 aa, Bifidobacterium bifidum
VEDATRSDSTTQMSSTPEVVYSSAVDSKQNRTSDFDANWKFMLSDSVQAQDPAFDDSAWQQVDLPHDYSITQKYSQSNEAESAYLPGGTGWYRKSFTIDRDLAGKRIAINFDGVYMNATVWFNGVKLGTHPYGYSPFSFDLTGNAKFGGENTIVVKVENRLPSSRWYSGSGIYRDVTLTVTDGVHVGNNGVAIKTPSLATQNGGDVTMNLTTKVANDTEAAANITLKQTVFPKGGKTDAAIGTVTTASKSIAAGASADVTSTITAASPKLWSIKNPNLYTVRTEVLNGGKVLDTYDTEYGFRWTGFDATSGFSLNGEKVKLKGVSMHHDQGSLGAVANRRAIERQVEILQKMGVNSIRTTHNPAAKALIDVCNEKGVLVVEEVFDMWNRSKNGNTEDYGKWFGQAIAGDNAVLGGDKDETWAKFDLTSTINRDRNAPSVIMWSLGNEMMEGISGSVSGFPATSAKLVAWTKAADSTRPMTYGDNKIKANWNESNTMGDNLTANGGVVGTNYSDGANYDKIRTTHPSWAIYGSETASAINSRGIYNRTTGGAQSSDKQLTSYDNSAVGWGAVASSAWYDVVQRDFVAGTYVWTGFDYLGEPTPWNGTGSGAVGSWPSPKNSYFGIVDTAGFPKDTYYFYQSQWNDDVHTLHILPAWNENVVAKGSGNNVPVVVYTDAAKVKLYFTPKGSTEKRLIGEKSFTKKTTAAGYTYQVYEGSDKDSTAHKNMYLTWNVPWAEGTISAEAYDENNRLIPEGSTEGNASVTTTGKAAKLKADADRKTITADGKDLSYIEVDVTDANGHIVPDAANRVTFDVKGAGKLVGVDNGSSPDHDSYQADNRKAFSGKVLAIVQSTKEAGEITVTAKADGLQSSTVKIATTAVPGTSTEKTVRSFYYSRNYYVKTGNKPILPSDVEVRYSDGTSDRQNVTWDAVSDDQIAKAGSFSVAGTVAGQKISVRVTMIDEIGALLNYSASTPVGTPAVLPGSRPAVLPDGTVTSANFAVHWTKPADTVYNTAGTVKVPGTATVFGKEFKVTATIRVQ

SEQ ID NO: 4 - protein, 1142 aa, Bifidobacterium bifidum
VEDATRSDSTTQMSSTPEVVYSSAVDSKQNRTSDFDANWKFMLSDSVQAQDPAFDDSAWQQVDLPHDYSITQKYSQSNEAESAYLPGGTGWYRKSFTIDRDLAGKRIAINFDGVYMNATVWFNGVKLGTHPYGYSPFSFDLTGNAKFGGENTIVVKVENRLPSSRWYSGSGIYRDVTLTVTDGVHVGNNGVAIKTPSLATQNGGDVTMNLTTKVANDTEAAANITLKQTVFPKGGKTDAAIGTVTTASKSIAAGASADVTSTITAASPKLWSIKNPNLYTVRTEVLNGGKVLDTYDTEYGFRWTGFDATSGFSLNGEKVKLKGVSMHHDQGSLGAVANRRAIERQVEILQKMGVNSIRTTHNPAAKALIDVCNEKGVLVVEEVFDMWNRSKNGNTEDYGKWFGQAIAGDNAVLGGDKDETWAKFDLTSTINRDRNAPSVIMWSLGNEMMEGISGSVSGFPATSAKLVAWTKAADSTRPMTYGDNKIKANWNESNTMGDNLTANGGVVGTNYSDGANYDKIRTTHPSWAIYGSETASAINSRGIYNRTTGGAQSSDKQLTSYDNSAVGWGAVASSAWYDVVQRDFVAGTYVWTGFDYLGEPTPWNGTGSGAVGSWPSPKNSYFGIVDTAGFPKDTYYFYQSQWNDDVHTLHILPAWNENVVAKGSGNNVPVVVYTDAAKVKLYFTPKGSTEKRLIGEKSFTKKTTAAGYTYQVYEGSDKDSTAHKNMYLTWNVPWAEGTISAEAYDENNRLIPEGSTEGNASVTTTGKAAKLKADADRKTITADGKDLSYIEVDVTDANGHIVPDAANRVTFDVKGAGKLVGVDNGSSPDHDSYQADNRKAFSGKVLAIVQSTKEAGEITVTAKADGLQSSTVKIATTAVPGTSTEKTVRSFYYSRNYYVKTGNKPILPSDVEVRYSDGTSDRQNVTWDAVSDDQIAKAGSFSVAGTVAGQKISVRVTMIDEIGALLNYSASTPVGTPAVLPGSRPAVLPDGTVTSANFAVHWTKPADTVYNTAGTVKVPGTATVFGKEFKVTATIRVQRSQVTIGSSVSGNALRLTQNIPADKQSDTLDAIKDGSTTVDANTGGGANPSAWTNWAYSKAGHNTAEITFEYATEQQLGQIVMYFFRDSNAVRFPDAGKTKIQI

SEQ ID NO: 5 - protein, 1211 aa, Bifidobacterium bifidum
VEDATRSDSTTQMSSTPEVVYSSAVDSKQNRTSDFDANWKFMLSDSVQAQDPAFDDSAWQQVDLPHDYSITQKYSQSNEAESAYLPGGTGWYRKSFTIDRDLAGKRIAINFDGVYMNATVWFNGVKLGTHPYGYSPFSFDLTGNAKFGGENTIVVKVENRLPSSRWYSGSGIYRDVTLTVTDGVHVGNNGVAIKTPSLATQNGGDVTMNLTTKVANDTEAAANITLKQTVFPKGGKTDAAIGTVTTASKSIAAGASADVTSTITAASPKLWSIKNPNLYTVRTEVLNGGKVLDTYDTEYGFRWTGFDATSGFSLNGEKVKLKGVSMHHDQGSLGAVANRRAIERQVEILQKMGVNSIRTTHNPAAKALIDVCNEKGVLVVEEVFDMWNRSKNGNTEDYGKWFGQAIAGDNAVLGGDKDETWAKFDLTSTINRDRNAPSVIMWSLGNEMMEGISGSVSGFPATSAKLVAWTKAADSTRPMTYGDNKIKANWNESNTMGDNLTANGGVVGTNYSDGANYDKIRTTHPSWAIYGSETASAINSRGIYNRTTGGAQSSDKQLTSYDNSAVGWGAVASSAWYDVVQRDFVAGTYVWTGFDYLGEPTPWNGTGSGAVGSWPSPKNSYFGIVDTAGFPKDTYYFYQSQWNDDVHTLHILPAWNENVVAKGSGNNVPVVVYTDAAKVKLYFTPKGSTEKRLIGEKSFTKKTTAAGYTYQVYEGSDKDSTAHKNMYLTWNVPWAEGTISAEAYDENNRLIPEGSTEGNASVTTTGKAAKLKADADRKTITADGKDLSYIEVDVTDANGHIVPDAANRVTFDVKGAGKLVGVDNGSSPDHDSYQADNRKAFSGKVLAIVQSTKEAGEITVTAKADGLQSSTVKIATTAVPGTSTEKTVRSFYYSRNYYVKTGNKPILPSDVEVRYSDGTSDRQNVTWDAVSDDQIAKAGSFSVAGTVAGQKISVRVTMIDEIGALLNYSASTPVGTPAVLPGSRPAVLPDGTVTSANFAVHWTKPADTVYNTAGTVKVPGTATVFGKEFKVTATIRVQRSQVTIGSSVSGNALRLTQNIPADKQSDTLDAIKDGSTTVDANTGGGANPSAWTNWAYSKAGHNTAEITFEYATEQQLGQIVMYFFRDSNAVRFPDAGKTKIQISADGKNWTDLAATETIAAQESSDRVKPYTYDFAPVGATFVKVTVTNADTTTPSGVVCAGLTEIELKTAT

SEQ ID NO: 6 - protein, 1025 aa, Kluyveromyces lactis
MSCLIPENLRNPKKVHENRLPTRAYYYDQDIFESLNGPWAFALFDAPLDAPDAKNLDWETAKKWSTISVPSHWELQEDWKYGKPIYTNVQYPIPIDIPNPPTVNPTGVYARTFELDSKSIESFEHRLRFEGVDNCYELYVNGQYVGFNKGSRNGAEFDIQKYVSEGENLVVVKVFKWSDSTYIEDQDQWWLSGIYRDVSLLKLPKKAHIEDVRVTTTFVDSQYQDAELSVKVDVQGSSYDHINFTLYEPEDGSKVYDASSLLNEENGNTTFSTKEFISFSTKKNEETAFKINVKAPEHWTAENPTLYKYQLDLIGSDGSVIQSIKHHVGFRQVELKDGNITVNGKDILFRGVNRHDHHPRFGRAVPLDFVVRDLILMKKFNINAVRNSHYPNHPKVYDLFDKLGFWVIDEADLETHGVQEPFNRHTNLEAEYPDTKNKLYDVNAHYLSDNPEYEVAYLDRASQLVLRDVNHPSIIIWSLGNEACYGRNHKAMYKLIKQLDPTRLVHYEGDLNALSADIFSFMYPTFEIMERWRKNHTDENGKFEKPLILCEYGHAMGNGPGSLKEYQELFYKEKFYQGGFIWEWANHGIEFEDVSTADGKLHKAYAYGGDFKEEVHDGVFIMDGLCNSEHNPTPGLVEYKKVIEPVHIKIAHGSVTITNKHDFITTDHLLFIDKDTGKTIDVPSLKPEESVTIPSDTTYVVAVLKDDAGVLKAGHEIAWGQAELPLKVPDFVTETAEKAAKINDGKRYVSVESSGLHFILDKLLGKIESLKVKGKEISSKFEGSSITFWRPPTNNDEPRDFKNWKKYNIDLMKQNIHGVSVEKGSNGSLAVVTVNSRISPVVFYYGFETVQKYTIFANKINLNTSMKLTGEYQPPDFPRVGYEFWLGDSYESFEWLGRGPGESYPDKKESQRFGLYDSKDVEEFVYDYPQENGNHTDTHFLNIKFEGAGKLSIFQKEKPFNFKISDEYGVDEAAHACDVKRYGRHYLRLDHAIHGVGSEACGPAVLDQYRLKAQDFNFEFDLAFE

SEQ ID NO: 7 - protein, 1008 aa, Lactobacillus delbrueckii subsp. bulgaricus
MSNKLVKEKRVDQADLAWLTDPEVYEVNTIPPHSDHESFQSQEELEEGKSSLVQSLDGDWLIDYAENGQGPVNFYAEDFDDSNFKSVKVPGNLELQGFGQPQYVNVQYPWDGSEEIFPPQIPSKNPLASYVRYFDLDEAFWDKEVSLKFDGAATAIYVWLNGHFVGYGEDSFTPSEFMVTKFLKKENNRLAVALYKYSSASWLEDQDFWRMSGLFRSVTLQAKPRLHLEDLKLTASLTDNYQKGKLEVEANIAYRLPNASFKLEVRDSEGDLVAEKLGPIRSEQLEFTLADLPVAAWSAEKPNLYQVRLYLYQAGSLLEVSRQEVGFRNFELKDGIMYLNGQRIVFKGANRHEFDSKLGRAITEEDMIWDIKTMKRSNINAVRCSHYPNQSLFYRLCDKYGLYVIDEANLESHGTWEKVGGHEDPSFNVPGDDQHWLGASLSRVKNMMARDKNHASILIWSLGNESYAGTVFAQMADYVRKADPTRVQHYEGVTHNRKFDDATQIESRMYVPAKVIEEYLTNKPAKPFISVEYAHAMGNSVGDLAAYTALEKYPHYQGGFIWDWIDQGLEKDGHLLYGGDFDDRPTDYEFCGNGLVFADRTESPKLANVKALYANLKLEVKDGQLFLKNDNLFTNSSSYYFLTSLLVDGKLTYQSRPLTFGLEPGESGTFALPWPEVADEKGEVVYRVTAHLKEDLPWADEGFTVAEAEEVAQKLPEFKPEGRPDLVDSDYNLGLKGNNFQILFSKVKGWPVSLKYAGREYLKRLPEFTFWRALTDNDRGAGYGYDLARWENAGKYARLKDISCEVKEDSVLVKTAFTLPVALKGDLTVTYEVDGRGKIAVTADFPGAEEAGLLPAFGLNLALPKELTDYRYYGLGPNESYPDRLEGNYLGIYQGAVKKNFSPYLRPQETGNRSKVRWYQLFDEKGGLEFTANGADLNLSALPYSAAQIEAADHAFELTNNYTWVRALSAQMGVGGDDSWGQKVHPEFCLDAQKARQLRLVIQPLLLK

(19) United States
(12) Patent Application Publication
(10) Pub. No.: US 2022/0325307 A1
(43) Pub. Date: Oct. 13, 2022

(54) USE OF LACTASE TO PROVIDE HIGH GOS FIBER LEVEL AND LOW LACTOSE LEVEL

(71) Applicant: DUPONT NUTRITION BIOSCIENCES APS, COPENHAGEN K (DK)

(72) Inventors: Jacob Flyvholm CRAMER, Palo Alto, CA (US); Tina Krogh JENSEN, Skanderborg (DK); Karina Hansen KJAER, Brabrand (DK); Collette LENTZ, Spring Hill, KS (US)

(21) Appl. No.: 17/297,055

(22) PCT Filed: Nov. 26, 2019
(86) PCT No.: PCT/US19/63163

§ 371 Date: May 26, 2021

Related U.S. Application Data
(60) Provisional application No. 62/774,476, filed on Dec. 3, 2018.

(51) Int. Cl. C12P 19/04; C12P 19/18; C12N 9/24; A23C 9/12; A23L 33/21

(52) U.S. Cl. CPC C12P 19/04; C12P 19/18; C12Y 302/01023; C12Y 302/01108; C12N 9/2402; A23C 9/1206; A23L 33/21; A23V 2002/00

(57) ABSTRACT

A method for providing a low lactose milk-based product having GOS fiber is provided in which a milk substrate having lactose is treated with a transgalactosylating enzyme to provide GOS fiber and remaining lactose; deactivating the transgalactosylating enzyme; contacting the milk-based substrate having GOS fiber with a lactase to degrade the remaining lactose to provide the low lactose milk-based product having GOS fiber and deactivating the lactase. Also provided are enzymes and GOS fiber and lactose amounts and stability.

### FIELD OF THE INVENTION

The present invention relates to a method for preparing a dairy product having a stable content of GOS fiber, and to a GOS fiber-enriched dairy product prepared by the method in which the lactose content has also been significantly reduced.

### BACKGROUND OF THE INVENTION

Galactooligosaccharides (GOS) are carbohydrates which are nondigestable in humans and animals comprising two or more galactose molecules, typically up to nine, linked by glycosidic bonds. GOS may also include one or more glucose molecules. One of the beneficial effects of GOS is its ability of acting as prebiotic compounds by selectively stimulating the proliferation of beneficial colonic microorganisms such as bacteria to give physiological benefits to the consumer. The established health effects have resulted in a growing interest in GOS as food ingredients for various types of food.

The enzyme β-galactosidase (EC 3.2.1.23) usually hydrolyses lactose to the monosaccharides D-glucose and D-galactose. In the normal enzyme reaction of β-galactosidases, the enzyme hydrolyses lactose and transiently binds the galactose monosaccharide in a galactose-enzyme complex that transfers galactose to the hydroxyl group of water, resulting in the liberation of D-galactose and D-glucose. However, at high lactose concentrations some β-galactosidases are able to transfer galactose to the hydroxyl groups of D-galactose or D-glucose in a process called transgalactosylation whereby galacto-oligosaccharides are produced. At high lactose concentrations some β-galactosidases are able to transfer galactose to the hydroxyl groups of lactose or higher order oligosaccharides.

Enzymes and methods for creating high levels of GOS have been developed. See, e.g., Polypeptides Having Transgalactosylating Activity, WO 2013/182686. In the context of diary applications for milk-based products such as yogurt, cheese and milk beverages, while production of GOS depletes the endogenous lactose sugar, lactose levels remain too high for individuals with lactose intolerance. It is estimated that some 70% of the world's population are lactose intolerant, i.e. suffer from digestive disorders if they consume lactose.

There is a continuing need for milk-based products that have high, stable levels of GOS but are sufficiently low in lactose such that they can be consumed by lactose intolerant individuals.

### SUMMARY OF THE INVENTION

A method is presented for preparing a low lactose milk-based product having GOS fiber, the method having the steps of providing a milk-based substrate comprising lactose; treating said milk-based substrate with a transgalactosylating enzyme to provide GOS fiber and remaining lactose; deactivating the transgalactosylating enzyme; contacting the milk-based substrate having GOS fiber with a lactase to degrade the remaining lactose to provide the low lactose milk-based product having GOS fiber; and deactivating the lactase. Optionally, the milk-based substrate has a lactose concentration of between 1-60% (w/w); or 2-50% (w/w), or 3-40% (w/w); or 4-30% (w/w). Optionally, the transgalactosylating enzyme is a truncated β-galactosidase from Bifidobacterium bifidum.

Optionally, the truncated β-galactosidase from Bifidobacterium bifidum is truncated on the C-terminus. Optionally, the truncated β-galactosidase from Bifidobacterium bifidum is a polypeptide having at least 70% sequence identity to SEQ ID. NO:1, SEQ ID NO:2, SEQ ID NO:3, SEQ ID NO:4 or SEQ ID NO:5 or to a transgalactosylase active fragment thereof. Optionally, the polypeptide has at least 80% sequence identity to SEQ ID. NO:1, SEQ ID NO:2, SEQ ID NO:3, SEQ ID NO:4 or SEQ ID NO:5 or to a transgalactosylase active fragment thereof. Optionally, the polypeptide has at least 90% sequence identity to SEQ ID. NO:1, SEQ ID NO:2, SEQ ID NO:3, SEQ ID NO:4 or SEQ ID NO:5 or to a transgalactosylase active fragment thereof.

Optionally, the polypeptide has at least 95% sequence identity to SEQ ID. NO:1, SEQ ID NO:2, SEQ ID NO:3, SEQ ID NO:4 or SEQ ID NO:5 or to a transgalactosylase active fragment thereof. Optionally, the polypeptide has at least 99% sequence identity to SEQ ID. NO:1, SEQ ID NO:2, SEQ ID NO:3, SEQ ID NO:4 or SEQ ID NO:5 or to a transgalactosylase active fragment thereof. Optionally, the polypeptide comprises a sequence according to SEQ ID. NO:1, SEQ ID NO:2, SEQ ID NO:3, SEQ ID NO:4 or SEQ ID NO:5 or to a transgalactosylase active fragment thereof. Optionally, the polypeptide comprises a sequence according to SEQ ID. NO:1 or to a transgalactosylase active fragment thereof. Optionally, the polypeptide comprises a sequence according to SEQ ID. NO:1.

Optionally, the deactivation of the transgalactosylating enzyme comprises heat treatment. Optionally, the heat treatment is from about 70° C. to 95° C. and for between about 5 minutes to 30 minutes. Optionally, the heat treatment is at about 95° C. for 5 to 30 minutes. Optionally, the heat treatment is from about 135° C. to about 150° C. for about 2. seconds to about 15 seconds.

Optionally, the lactase is a K. locus lactase. Optionally, the K. lactic lactase is a polypeptide having at least 80% sequence identity to SEQ ID. NO. 6 or to a lactase active fragment thereof. Optionally, the K. lactis lactase is a polypeptide having at least 90% sequence identity to SEQ ID. NO. 6 or to a lactase active fragment thereof. Optionally, the K. lactis lactase is a polypeptide having at least 95% sequence identity to SEQ ID. NO. 6 or to a lactase active fragment thereof. Optionally, the K. lactis lactase is a polypeptide having at least 99% sequence identity to SEQ ID. NO. 6 or to a lactase active fragment thereof. Optionally, the K. lactis lactase is a polypeptide according to SEQ ID. NO. 6 or to a lactase active fragment thereof. Optionally, the K. lactis lactase is a polypeptide according to SEQ ID. NO. 6.

Optionally, less than 20% of the GOS fiber is hydrolyzed by the lactase during the step of contacting the milk-based substrate having GOS fiber with said lactase. Optionally, less than about 15% of the GOS fiber is hydrolyzed. Optionally, less than about 10% of the GOS fiber is hydrolyzed. Optionally, less than about 5% of the GOS fiber is hydrolyzed.

Optionally, the deactivation of the lactase enzyme comprises heat treatment. Optionally, the heat treatment is at about 135° C. to about 150° C. for about 2 to about 15 seconds, about 85° C. to about 115° C. for about 0.5 to about 9 seconds or at about 70° C. to about 85° C. for about 15 seconds to about 30 seconds.

Optionally, the deactivating step of the lactase comprises reduction of the pH of the milk-based substrate having GOS fiber and the lactase. Optionally, the pH is accomplished by adding yogurt or cheese cultures to the milk-based substrate.

Optionally, the low lactose milk-based product having GOS fiber is yoghurt, ice cream, UHT milk, flavored milk product, concentrated/condensed milk product, milk-based powder, or cheese. Optionally, the GOS fiber in the low lactose milk-based product is stable having a variance of :less than about 10% within 28 days.

Optionally, the low lactose milk-based product having GOS fiber contains more than about 1.5 (w/w) GOS fiber. Optionally, the low lactose milk-based product having GOS fiber contains more than about 3.2% (w/w) GOS fiber. Optionally, the low lactose milk-based product having GOS fiber contains more than about 4% (w/w) GOS fiber. Optionally, the low lactose milk-based product having GOS fiber contains more than about 7% (w/w) GOS fiber. Optionally, the low lactose milk-based product having GOS fiber contains more than about 14% (w/w) GOS fiber. Optionally, the low lactose milk-based product having GOS fiber contains more than about 30% (w/w) GOS fiber.

Optionally, the low lactose milk-based product having GOS fiber contains more than 1.5g GOS fiber per 100 kcal and below 0.1% lactose or below 0.01% lactose. Optionally, the lactose in the milk-based substrate has been reduced more than about 50%, more than about 97%, more than about 98%, more than about 99% or more than about 99.7%.

Optionally, the method further comprises the steps of dehydrating the low lactose milk-based product to provide a powder and dissolving the powder in water.

### BRIEF DESCRIPTION OF THE DRAWINGS

FIG. 1. depicts GOS stability as GOS was quantified according to example 4 at indicated days of storage at 5° C. relative to day 0.

FIG. 2. depicts GOS stability as GOS was quantified according to example 2 at indicated days of storage at relative to day 0 of a milk after enzyme treatment of 24 hours and inactivation by UHT pasteurization.

### BRIEF DESCRIPTION OF THE SEQUENCE IDs

SEQ ID NO:1 sets forth the amino acid sequence of BIF917 β-galactosidase.

SEQ ID NO:2 sets forth the amino acid sequence of BIF995 β-galactosidase.

SEQ ID NO:3 sets forth the amino acid sequence of BIF1068 β-galactosidase.

SEQ ID NO:4 sets forth the amino acid sequence of BIF1172 β-galactosidase.

SEQ ID NO:5 sets forth the amino acid sequence of BIF1241 β-galactosidase.

SEQ ID NO:6 sets forth the amino acid sequence of the mature form of B-galactosidase from Kluyveromyces lactis, KLac.

SEQ ID NO: 7 sets forth the amino acid sequence of the mature form of B-galactosidase from Lactobacillus delbrueckii bulgaricus, LBul.

### DETAILED DISCLOSURE OF THE INVENTION

### Detailed Description of the Inventions:

The practice of the present teachings will employ, unless otherwise indicated, conventional techniques of molecular biology (including recombinant techniques), microbiology, cell biology, and biochemistry, which are within the skill of the art. Such techniques are explained fully in the literature, for example, Molecular Cloning: A Laboratory Manual, second edition (Sambrook et al., 1989); Oligonucleotide Synthesis (M. J. Gait, ed., 1984; Current Protocols in Molecular Biology (F. M. Ausubel et al., eds., 1994); PCR: The Polymerase Chain Reaction (Mullis et al., eds., 1994); Gene Transfer and Expression: A Laboratory Manual (Kriegler, 1990), and The Alcohol Textbook (Ingledew et al., eds., Fifth Edition, 2009), and Essentials of Carbohydrate Chemistry and Biochemistry (Lindhorste, 2007).

Unless defined otherwise herein, all technical and scientific terms used herein have the same meaning as commonly understood by one of ordinary skill in the art to which the present teachings belong. Singleton, et al., Dictionary of Microbiology and Molecular Biology, second ed., John Wiley and Sons, New York (1994), and Hale & Markham, The Harper Collins Dictionary of Biology, Harper Perennial, NY (1991) provide one of skill with a general dictionary of many of the terms used in this invention. Any methods and materials similar or equivalent to those described herein can be used in the practice or testing of the present teachings.

Numeric ranges provided herein are inclusive of the numbers defining the range.

### Definitions:

A “β-galactosidase” is glycoside hydrolase that catalyzes the hydrolysis of β-galactosidase, including lactose, into monosaccharides. A β-galactosidase is also sometimes called a “lactase.”

The term “galactooligosaccharide” also referred to herein as “GOS” refers to nondigestable oligosaccharides composed of from 2 to 20 molecules of predominantly galactose. GOS is typically formed by β-galactosidase enzymes, also called lactases by degrading lactose in e.g. milk and/or milk-based products.

The term “GOS fiber” herein refers to nondigestable galactooligosaccharides with a degree of polymerization of 3 or more (DP3+).

The terms, “wild-type,” “parental,” or “reference,” with respect to a polypeptide, refer to a naturally-occurring polypeptide that does not include a man-made substitution, insertion, or deletion at one or more amino acid positions. Similarly, the terms “wild-type,” “parental,” or “reference,” with respect to a polynucleotide, refer to a naturally-occurring polynucleotide that does not include a man-made nucleotide change. However, note that a polynucleotide encoding a wild-type, parental, or reference polypeptide is not limited to a naturally-occurring polynucleotide, and encompasses any polynucleotide encoding the wild-type, parental, or reference polypeptide.

Reference to the wild-type polypeptide is understood to include the mature form of the polypeptide. A “mature” polypeptide or variant, thereof, is one in which a signal sequence is absent, for example, cleaved from an immature form of the polypeptide during or following expression of the polypeptide.

The term “variant,” with respect to a polypeptide, refers to a polypeptide that differs from a specified wild-type, parental, or reference polypeptide in that it includes one or more naturally-occurring or man-made substitutions, insertions, or deletions of an amino acid. Similarly, the term “variant,” with respect to a polynucleotide, refers to a polynucleotide that differs in nucleotide sequence from a specified wild-type, parental, or reference polynucleotide. The identity of the wild-type, parental, or reference polypeptide or polynucleotide will be apparent from context.

The term “recombinant,” when used in reference to a subject cell, nucleic acid, protein or vector, indicates that the subject has been modified from its native state. Thus, for example, recombinant cells express genes that are not found within the native (non-recombinant) form of the cell, or express native genes at different levels or under different conditions than found in nature. Recombinant nucleic acids differ from a native sequence by one or more nucleotides and/or are operably linked to heterologous sequences, e.g., a heterologous promoter in an expression vector. Recombinant proteins may differ from a native sequence by one or more amino acids and/or are fused with heterologous sequences. A vector comprising a nucleic acid encoding a β-galactosidase is a recombinant vector.

The terms “recovered,” “isolated,” and “separated,” refer to a compound, protein (polypeptides), cell, nucleic acid, amino acid, or other specified material or component that is removed from at least one other material or component with which it is naturally associated as found in nature. An “isolated” polypeptides, thereof, includes, but is not limited to, a culture broth containing secreted polypeptide expressed in a heterologous host cell.

The term “polymer” refers to a series of monomer groups linked together. A polymer is composed of multiple units of a single monomer. As used herein the term “glucose polymer” refers to glucose units linked together as a polymer. As long as there are at least three glucose units, the glucose polymer may contain non-glucose sugars such as lactose or galactose.

The term “amino acid sequence” is synonymous with the terms “polypeptide,” “protein,” and “peptide,” and are used interchangeably. Where such amino acid sequences exhibit activity, they may be referred to as an “enzyme.” The conventional one-letter or three-letter codes for amino acid residues are used, with amino acid sequences being presented in the standard amino-to-carboxy terminal orientation (i.e., N→C).

The term “nucleic acid” encompasses DNA, RNA, heteroduplexes, and synthetic molecules capable of encoding a polypeptide. Nucleic acids may be single stranded or double stranded and may be chemically modified. The terms “nucleic acid” and “polynucleotide” are used interchangeably. Because the genetic code is degenerate, more than one codon may be used to encode a particular amino acid, and the present compositions and methods encompass nucleotide sequences that encode a particular amino acid sequence. Unless otherwise indicated, nucleic acid sequences are presented in 5′-to-3′ orientation.

The terms “transformed,” “stably transformed,” and “transgenic,” used with reference to a cell means that the cell contains a non-native (e.g., heterologous) nucleic acid sequence integrated into its genome or carried as an episome that is maintained through multiple generations.

The term “introduced” in the context of inserting a nucleic acid sequence into a cell, means “transfection”, “transformation” or “transduction,” as known in the art.

A “host strain” or “host cell” is an organism into which an expression vector, phage, virus, or other DNA construct, including a polynucleotide encoding a polypeptide of interest (e.g., a β-galactosidase) has been introduced. Exemplary host strains are microorganism cells (e.g., bacteria, filamentous fungi, and yeast) capable of expressing the polypeptide of interest. The term “host cell” includes protoplasts created from cells.

The term “heterologous” with reference to a polynucleotide or protein refers to a polynucleotide or protein that does not naturally occur in a host cell.

The term “endogenous” with reference to a polynucleotide or protein refers to a polynucleotide or protein that occurs naturally in the host cell.

The term “expression” refers to the process by which a polypeptide is produced based on a nucleic acid sequence. The process includes both transcription and translation.

A “selective marker” or “selectable marker” refers to a gene capable of being expressed in a host to facilitate selection of host cells carrying the gene. Examples of selectable markers include but are not limited to antimicrobials (e.g., hygromycin, bleomycin, or chloramphenicol) and/or genes that confer a metabolic advantage, such as a nutritional advantage on the host cell.

A “vector” refers to a polynucleotide sequence designed to introduce nucleic acids into one or more cell types. Vectors include cloning vectors, expression vectors, shuttle vectors, plasmids, phage particles, cassettes and the like.

An “expression vector” refers to a DNA construct comprising a DNA sequence encoding a polypeptide of interest, which coding sequence is operably linked to a suitable control sequence capable of effecting expression of the DNA in a suitable host. Such control sequences may include a promoter to effect transcription, an optional operator sequence to control transcription, a sequence encoding suitable ribosome binding sites on the mRNA, enhancers and sequences which control termination of transcription and translation.

The term “operably linked” means that specified components are in a relationship (including but not limited to juxtaposition) permitting them to function in an intended manner. For example, a regulatory sequence is operably linked to a coding sequence such that expression of the coding sequence is under control of the regulatory sequences.

A “signal sequence” is a sequence of amino acids attached to the N-terminal portion of a protein, which facilitates the secretion of the protein outside the cell. The mature form of an extracellular protein lacks the signal sequence, which is cleaved off during the secretion process.

“Biologically active” refers to a sequence having a specified biological activity, such an enzymatic activity.

The term “specific activity” refers to the number of moles of substrate that can be converted to product by an enzyme or enzyme preparation per unit time under specific conditions. Specific activity is generally expressed as units (U)/mg of protein.

As used herein, “percent sequence identity” means that a particular sequence has at least a certain percentage of amino acid residues identical to those in a specified reference sequence, when aligned using the CLUSTAL W algorithm with default parameters. See Thompson et al. (1994) Nucleic Acids Res. 22:4673-4680. Default parameters for the CLUSTAL W algorithm are:
Gap opening penalty: 10.0
Gap extension penalty: 0.05
Protein weight matrix: BLOSUM series
DNA weight matrix: TUB
Delay divergent sequences %: 40
Gap separation distance: 8
DNA transitions weight: 0.50
List hydrophilic residues: GPSNDQEKR
Use negative matrix: OFF
Toggle Residue specific penalties: ON
Toggle hydrophilic penalties: ON
Toggle end gap separation penalty OFF.

Deletions are counted as non-identical residues, compared to a reference sequence. Deletions occurring at either terminus are included. For example, a variant with five amino acid deletions of the C-terminus of the mature 617 residue polypeptide would have a percent sequence identity of 99% (612/617 identical residues×100, rounded to the nearest whole number) relative to the mature polypeptide. Such a variant would be encompassed by a variant having “at least 99% sequence identity” to a mature polypeptide.

“Fused” polypeptide sequences are connected, i.e., operably linked, via a peptide bond between two subject polypeptide sequences.

The term “filamentous fungi” refers to all filamentous forms of the subdivision Eumycotina, particularly Pezizomycotina species.

The term “about” refers to ±5% to the referenced value. “Lactase treated milk” means milk treated with lactase to reduce the amount of lactose sugar.

“Reduced lactose milk” means milk wherein the percentage of lactose is about 2% or lower.

“Lactose free milk” means milk wherein the percentage of lactose is below 0.1% (w/v) and in some instances this would mean milk wherein the percentage of lactose is below 0.01% (w/v).

### Additional Mutations

In some embodiments, the present β-galactosidases further include one or more mutations that provide a further performance or stability benefit. Exemplary performance benefits include but are not limited to increased thermal stability, increased storage stability, increased solubility, an altered pH profile, increased specific activity, modified substrate specificity, modified substrate binding, modified pH-dependent activity, modified pH-dependent stability, increased oxidative stability, and increased expression. In some cases, the performance benefit is realized at a relatively low temperature. In some cases, the performance benefit is realized at relatively high temperature.

Furthermore, the present β-galactosidases may include any number of conservative amino acid substitutions. Exemplary conservative amino acid substitutions are listed in the following Table.

### Conservative Amino Acid Substitutions

| For Amino Acid | Code | Replace with any of |
|---|---|---|
| Alanine | A | D-Ala, Gly, beta-Ala, L-Cys, D-Cys |
| Arginine | R | D-Arg, Lys, D-Lys, homo-Arg, D-homo-Arg, |
| | | Met, Ile, D-Met, D-Ile, Orn, D-Orn |
| Asparagine | N | D-Asn, Asp, D-Asp, Glu, D-Glu, Gln, D-Gln |
| Aspartic Acid | D | D-Asp, D-Asn, Asn, Glu, D-Glu, Gln, D-Gln |
| Cysteine | C | D-Cys, S—Me-Cys, Met, D-Met, Thr, D-Thr |
| Glutamine | Q | D-Gln, Asn, D-Asn, Glu, D-Glu, Asp, D-Asp |
| Glutamic Acid | E | D-Glu, D-Asp, Asp, Asn, D-Asn, Gln, D-Gln |
| Glycine | G | Ala, D-Ala, Pro, D-Pro, b-Ala, Acp |
| Isoleucine | I | D-Ile, Val, D-Val, Leu, D-Leu, Met, D-Met |
| Leucine | L | D-Leu, Val, D-Val, Leu, D-Leu, Met, D-Met |
| Lysine | K | D-Lys, Arg, D-Arg, homo-Arg, D-homo-Arg, |
| | | Met, D-Met, Ile, D-Ile, Orn, D-Orn |
| Methionine | M | D-Met, S—Me-Cys, Ile, D-Ile, Leu, D-Leu, |
| | | Val, D-Val |
| Phenylalanine | F | D-Phe, Tyr, D-Thr, L-Dopa, His, D-His, Trp, |
| | | D-Trp, Trans-3,4, or 5-phenylproline, cis-3,4, |
| | | or 5-phenylproline |
| Proline | P | D-Pro, L-I-thioazolidine-4-carboxylic acid, |
| | | D-or L-1-oxazolidine-4-carboxylic acid |
| Serine | S | D-Ser, Thr, D-Thr, allo-Thr, Met, D-Met, |
| | | Met(O), D-Met(O), L-Cys, D-Cys |
| Threonine | T | D-Thr, Ser, D-Ser, allo-Thr, Met, |
| | | D-Met, Met(O), D-Met(O), Val, D-Val |
| Tyrosine | Y | D-Tyr, Phe, D-Phe, L-Dopa, His, D-His |
| Valine | V | D-Val, Leu, D-Leu, Ile, D-Ile, Met, D-Met |

The reader will appreciate that some of the above mentioned conservative mutations can be produced by genetic manipulation, while others are produced by introducing synthetic amino acids into a polypeptide by genetic or other means.

The present β-galactosidases may be “precursor,” “immature,” or “full-length,” in which case they include a signal sequence, or “mature,” in which case they lack a signal sequence. Mature forms of the polypeptides are generally the most useful. Unless otherwise noted, the amino acid residue numbering used herein refers to the mature forms of the respective β-galactosidase polypeptides. The present β-galactosidase polypeptides may also be truncated to remove the N or C-termini, so long as the resulting polypeptides retain β-galactosidase activity.

The present β-galactosidases may be a “chimeric” or “hybrid” polypeptide, in that it includes at least a portion of a first β-galactosidase polypeptide, and at least a portion of a second β-galactosidase polypeptide. The present β-galactosidases may further include heterologous signal sequence, an epitope to allow tracking or purification, or the like. Exemplary heterologous signal sequences are from B. licheniformis amylase (LAT), B. subtilis (AmyE or AprE), and Streptomyces CelA.

Production of β-galactosidases

The present β-galactosidases can be produced in host cells, for example, by secretion or intracellular expression. A cultured cell material (e.g., a whole-cell broth) comprising a β-galactosidase can be obtained following secretion of the β-galactosidase into the cell medium. Optionally, the β-galactosidase can be isolated from the host cells, or even isolated from the cell broth, depending on the desired purity of the final β-galactosidase. A gene encoding a β-galactosidase can be cloned and expressed according to methods well known in the art. Suitable host cells include bacterial, fungal (including yeast and filamentous fungi), and plant cells (including algae). Particularly useful host cells include Aspergillus niger, Aspergillus oryzae or Trichoderma reesei. Other host cells include bacterial cells, e.g., Bacillus subtilis or B. licheniformis, as well as Streptomyces, and E. Coli.

The host cell further may express a nucleic acid encoding a homologous or heterologous β-galactosidase, i.e., a β-galactosidase that is not the same species as the host cell, or one or more other enzymes. The β-galactosidase may be a variant β-galactosidase. Additionally, the host may express one or more accessory enzymes, proteins, peptides. Vectors

A DNA construct comprising a nucleic acid encoding a β-galactosidase can be constructed to be expressed in a host cell. Because of the well-known degeneracy in the genetic code, variant polynucleotides that encode an identical amino acid sequence can be designed and made with routine skill. It is also well-known in the art to optimize codon use for a particular host cell. Nucleic acids encoding β-galactosidase can be incorporated into a vector. Vectors can be transferred to a host cell using well-known transformation techniques, such as those disclosed below.

The vector may be any vector that can be transformed into and replicated within a host cell. For example, a vector comprising a nucleic acid encoding a β-galactosidase can be transformed and replicated in a bacterial host cell as a means of propagating and amplifying the vector. The vector also may be transformed into an expression host, so that the encoding nucleic acids can be expressed as a functional β-galactosidase. Host cells that serve as expression hosts can include filamentous fungi, for example.

A nucleic acid encoding a β-galactosidase can be operably linked to a suitable promoter, which allows transcription in the host cell. The promoter may be any DNA sequence that shows transcriptional activity in the host cell of choice and may be derived from genes encoding proteins either homologous or heterologous to the host cell. Exemplary promoters for directing the transcription of the DNA sequence encoding a β-galactosidase, especially in a bacterial host, are the promoter of the lac operon of E. coli, the Streptomyces coelicolor agarase gene dagA or celA promoters, the promoters of the Bacillus licheniformis α-amylase gene (amyL), the promoters of the Bacillus stearothermophilus maltogenic amylase gene (amyM), the promoters of the Bacillus amyloliquefaciens α-amylase (amyQ), the promoters of the Bacillus subtilis xylA and xylB genes etc. For transcription in a fungal host, examples of useful promoters are those derived from the gene encoding Aspergillus oryzae TAKA amylase, Rhizomucor miehei aspartic proteinase, Aspergillus niger neutral α-amylase, A. niger acid stable α-amylase, A. niger glucoamylase, Rhizomucor miehei lipase, A. oryzae alkaline protease, A. oryzae triose phosphate isomerase, or A. nidulans acetamidase. When a gene encoding a β-galactosidase is expressed in a bacterial species such as E. coli, a suitable promoter can be selected, for example, from a bacteriophage promoter including a T7 promoter and a phage lambda promoter. Examples of suitable promoters for the expression in a yeast species include but are not limited to the Gal 1 and Gal 10 promoters of Saccharomyces cerevisiae and the Pichia pastoris AOX1 or AOX2 promoters. cbh1 is an endogenous, inducible promoter from T. reesei. See Liu et al. (2008) “Improved heterologous gene expression in Trichoderma reesei by cellobiohydrolase I gene (cbh1) promoter optimization,” Acta Biochim. Biophys. Sin (Shanghai) 40(2): 158-65.

The coding sequence can be operably linked to a signal sequence. The DNA encoding the signal sequence may be the DNA sequence naturally associated with the β-galactosidase gene to be expressed or from a different Genus or species. A signal sequence and a promoter sequence comprising a DNA construct or vector can be introduced into a fungal host cell and can be derived from the same source. For example, the signal sequence is the cbh1 signal sequence that is operably linked to a cbh1 promoter.

An expression vector may also comprise a suitable transcription terminator and, in eukaryotes, polyadenylation sequences operably linked to the DNA sequence encoding a variant β-galactosidase. Termination and polyadenylation sequences may suitably be derived from the same sources as the promoter.

The vector may further comprise a DNA sequence enabling the vector to replicate in the host cell. Examples of such sequences are the origins of replication of plasmids pUC19, pACYC177, pUB110, pE194, pAMB1, and pIJ702.

The vector may also comprise a selectable marker, e.g., a gene the product of which complements a defect in the isolated host cell, such as the dal genes from B. subtilis or B. licheniformis, or a gene that confers antibiotic resistance such as, e.g., ampicillin, kanamycin, chloramphenicol, or tetracycline resistance. Furthermore, the vector may comprise Aspergillus selection markers such as amdS, argB, niaD and xxsC, a marker giving rise to hygromycin resistance, or the selection may be accomplished by co-transformation, such as known in the art. See e.g., International PCT Application WO 91/17243.

Intracellular expression may be advantageous in some respects, e.g., when using certain bacteria or fungi as host cells to produce large amounts of β-galactosidase for subsequent enrichment or purification. Extracellular secretion of β-galactosidase into the culture medium can also be used to make a cultured cell material comprising the isolated β-galactosidase.

The expression vector typically includes the components of a cloning vector, such as, for example, an element that permits autonomous replication of the vector in the selected host organism and one or more phenotypically detectable markers for selection purposes. The expression vector normally comprises control nucleotide sequences such as a promoter, operator, ribosome binding site, translation initiation signal and optionally, a repressor gene or one or more activator genes. Additionally, the expression vector may comprise a sequence coding for an amino acid sequence capable of targeting the β-galactosidase to a host cell organelle such as a peroxisome, or to a particular host cell compartment. Such a targeting sequence includes but is not limited to the sequence, SKL. For expression under the direction of control sequences, the nucleic acid sequence of the β-galactosidase is operably linked to the control sequences in proper manner with respect to expression.

The procedures used to ligate the DNA construct encoding a β-galactosidase, the promoter, terminator and other elements, respectively, and to insert them into suitable vectors containing the information necessary for replication, are well known to persons skilled in the art (see, e.g., Sambrook et al., MOLECULAR CLONING: A LABORATORY MANUAL, 2^{nd} ed., Cold Spring Harbor, 1989, and 3^{rd} ed., 2001).

### Transformation and Culture of Host Cells

An isolated cell, either comprising a DNA construct or an expression vector, is advantageously used as a host cell in the recombinant production of a β-galactosidase. The cell may be transformed with the DNA construct encoding the enzyme, conveniently by integrating the DNA construct (in one or more copies) in the host chromosome. This integration is generally considered to be an advantage, as the DNA sequence is more likely to be stably maintained in the cell. Integration of the DNA constructs into the host chromosome may be performed according to conventional methods, e.g., by homologous or heterologous recombination. Alternatively, the cell may be transformed with an expression vector as described above in connection with the different types of host cells.

Examples of suitable bacterial host organisms are Gram positive bacterial species such as Bacillaceae including Bacillus subtilis, Bacillus licheniformis, Bacillus lentus, Bacillus brevis, Geobacillus (formerly Bacillus) stearothermophilus, Bacillus alkalophilus, Bacillus amyloliquefaciens, Bacillus coagulans, Bacillus lautus, Bacillus megaterium, and Bacillus thuringiensis; Streptomyces species such as Streptomyces murinus; lactic acid bacterial species including Lactococcus sp. such as Lactococcus lactis; Lactobacillus sp. including Lactobacillus reuteri; Leuconostoc sp.; Pediococcus sp.; and Streptococcus sp. Alternatively, strains of a Gram negative bacterial species belonging to Enterobacteriaceae including E. coli, or to Pseudomonadaceae can be selected as the host organism.

A suitable yeast host organism can be selected from the biotechnologically relevant yeasts species such as but not limited to yeast species such as Pichia sp., Hansenula sp., or Kluyveromyces, Yarrowinia, Schizosaccharomyces species or a species of Saccharomyces, including Saccharomyces cerevisiae or a species belonging to Schizosaccharomyces such as, for example, S. pombe species. A strain of the methylotrophic yeast species, Pichia pastoris, can be used as the host organism. Alternatively, the host organism can be a Hansenula species. Suitable host organisms among filamentous fungi include species of Aspergillus, e.g., Aspergillus niger, Aspergillus oryzae, Aspergillus tubigensis, Aspergillus awamori, or Aspergillus nidulans. Alternatively, strains of a Fusarium species, e.g., Fusarium oxysporum or of a Rhizomucor species such as Rhizomucor miehei can be used as the host organism. Other suitable strains include Thermomyces and Mucor species. In addition, Trichoderma sp. can be used as a host. A suitable procedure for transformation of Aspergillus host cells includes, for example, that described in EP 238023. A β-galactosidase expressed by a fungal host cell can be glycosylated, i.e., will comprise a glycosyl moiety. The glycosylation pattern can be the same or different as present in the wild-type β-galactosidase. The type and/or degree of glycosylation may impart changes in enzymatic and/or biochemical properties.

It is advantageous to delete genes from expression hosts, where the gene deficiency can be cured by the transformed expression vector. Known methods may be used to obtain a fungal host cell having one or more inactivated genes. Gene inactivation may be accomplished by complete or partial deletion, by insertional inactivation or by any other means that renders a gene nonfunctional for its intended purpose, such that the gene is prevented from expression of a functional protein. A gene from a Trichoderma sp. or other filamentous fungal host that has been cloned can be deleted, for example, cbh1, cbh2, egl1, and egl2 genes. Gene deletion may be accomplished by inserting a form of the desired gene to be inactivated into a plasmid by methods known in the art.

Introduction of a DNA construct or vector into a host cell includes techniques such as transformation; electroporation; nuclear microinjection; transduction; transfection, e.g., lipofection mediated and DEAE-Dextrin mediated transfection; incubation with calcium phosphate DNA precipitate; high velocity bombardment with DNA-coated microprojectiles; and protoplast fusion. General transformation techniques are known in the art. See, e.g., Sambrook et al. (2001), supra. The expression of heterologous protein in Trichoderma is described, for example, in U.S. Pat. No. 6,022,725. Reference is also made to Cao et al. (2000) Science 9:991-1001 for transformation of Aspergillus strains. Genetically stable transformants can be constructed with vector systems whereby the nucleic acid encoding a β-galactosidase is stably integrated into a host cell chromosome. Transformants are then selected and purified by known techniques.

### Expression

A method of producing a β-galactosidase may comprise cultivating a host cell as described above under conditions conducive to the production of the enzyme and recovering the enzyme from the cells and/or culture medium.

The medium used to cultivate the cells may be any conventional medium suitable for growing the host cell in question and obtaining expression of a β-galactosidase. Suitable media and media components are available from commercial suppliers or may be prepared according to published recipes (e.g., as described in catalogues of the American Type Culture Collection).

An enzyme secreted from the host cells can be used in a whole broth preparation. In the present methods, the preparation of a spent whole fermentation broth of a recombinant microorganism can be achieved using any cultivation method known in the art resulting in the expression of a β-galactosidase. Fermentation may, therefore, be understood as comprising shake flask cultivation, small- or large-scale fermentation (including continuous, batch, fed-batch, or solid state fermentations) in laboratory or industrial fermenters performed in a suitable medium and under conditions allowing the β-galactosidase to be expressed or isolated. The term “spent whole fermentation broth” is defined herein as unfractionated contents of fermentation material that includes culture medium, extracellular proteins (e.g., enzymes), and cellular biomass. It is understood that the term “spent whole fermentation broth” also encompasses cellular biomass that has been lysed or permeabilized using methods well known in the art.

An enzyme secreted from the host cells may conveniently be recovered from the culture medium by well-known procedures, including separating the cells from the medium by centrifugation or filtration, and precipitating proteinaceous components of the medium by means of a salt such as ammonium sulfate, followed by the use of chromatographic procedures such as ion exchange chromatography, affinity chromatography, or the like.

The polynucleotide encoding a β-galactosidase in a vector can be operably linked to a control sequence that is capable of providing for the expression of the coding sequence by the host cell, i.e. the vector is an expression vector. The control sequences may be modified, for example by the addition of further transcriptional regulatory elements to make the level of transcription directed by the control sequences more responsive to transcriptional modulators. The control sequences may in particular comprise promoters.

Host cells may be cultured under suitable conditions that allow expression of a β-galactosidase. Expression of the enzymes may be constitutive such that they are continually produced, or inducible, requiring a stimulus to initiate expression. In the case of inducible expression, protein production can be initiated when required by, for example, addition of an inducer substance to the culture medium, for example dexamethasone or IPTG or Sophorose. Polypeptides can also be produced recombinantly in an in vitro cell-free system, such as the TNT™ (Promega) rabbit reticulocyte system.

Methods for Enriching and Purifying β-galactosidases

Fermentation, separation, and concentration techniques are well known in the art and conventional methods can be used in order to prepare a β-galactosidase polypeptide-containing solution.

After fermentation, a fermentation broth is obtained, the microbial cells and various suspended solids, including residual raw fermentation materials, are removed by conventional separation techniques in order to obtain a β-galactosidase solution. Filtration, centrifugation, microfiltration, rotary vacuum drum filtration, ultrafiltration, centrifugation followed by ultra-filtration, extraction, or chromatography, or the like, are generally used.

It is desirable to concentrate a β-galactosidase polypeptide-containing solution in order to optimize recovery. Use of unconcentrated solutions requires increased incubation time in order to collect the enriched or purified enzyme precipitate.

The enzyme containing solution is concentrated using conventional concentration techniques until the desired enzyme level is obtained. Concentration of the enzyme containing solution may be achieved by any of the techniques discussed herein. Exemplary methods of enrichment and purification include but are not limited to rotary drum vacuum filtration and/or ultrafiltration.

The β-galactosidases in certain embodiments of the invention may be derived from a Streptococcus species, Leuconostoc species or Lactobacillus species, for example. Examples of Streptococcus species from which the β-galactosidase may be derived include S. salivarius, S. sobrinus, S. dentirousetti, S. downei, S. mutans, S. oralis, S. gallolyticus and S. sanguinis. Examples of Leuconostoc species from which the β-galactosidase may be derived include L. mesenteroides, L. amelibiosum, L. argentinum, L. carnosum, L. citreum, L. cremoris, L. dextranicum and L. fructosum. Examples of Lactobacillus species from which the β-galactosidase may be derived include L. acidophilus, L. delbrueckii, L. helveticus, L. salivarius, L. casei, L. curvatus, L. plantarum, L. sakei, L. brevis, L. buchneri, L. fermentum and L. reuteri.

### Pasteurization Time and Temperatures

Pasteurization of the milk-base at: ultra-high temperature (UHT) is normally carried out at 135-150° C. for 2-15 sec, higher-heat/shorter time (HHST) normally carried out at 85-115° C. for 0.5-9 sec and High Temperature/Short Time (HTST) pasteurization normally carried out at 70-85° C. for 15-30sek.

### Preferred Embodiments of the Invention

In accordance with an aspect of the present invention, a method is presented for preparing a low lactose milk-based product having GOS fiber, the method having the steps of providing a milk-based substrate comprising lactose; treating said milk-based substrate with a transgalactosylating enzyme to provide GOS fiber and remaining lactose; deactivating the transgalactosylating enzyme; and contacting the milk-based substrate having GOS fiber with a lactase to degrade the remaining lactose to provide the low lactose milk-based product having GOS fiber; and deactivating the lactase.

Preferably, the milk-based substrate has a lactose concentration of between 1-60% (w/w); or 2-50% (w/w), or 3-40% (w/w); or 4-30% (w/w).

Preferably, the transgalactosylating enzyme is a truncated β-galactosidase from Bifidobacterium bifidum. More preferably, the truncated β-galactosidase from Bifidobacterium bifidum is truncated on the C-terminus. Still more preferably, the truncated β-galactosidase from Bifidobacterium bifidum is a polypeptide having at least 70% sequence identity to SEQ ID. NO:1, SEQ ID NO:2, SEQ ID NO:3, SEQ ID NO:4 or SEQ ID NO:5 or to a transgalactosylase active fragment thereof. In still more preferred embodiments, the polypeptide has at least 80% sequence identity to SEQ ID. NO:1, SEQ ID NO:2, SEQ ID NO:3, SEQ ID NO:4 or SEQ ID NO:5 or to a transgalactosylase active fragment thereof. Yet more preferably, the polypeptide has at least 90% sequence identity to SEQ ID. NO:1, SEQ ID NO:2, SEQ ID NO:3, SEQ ID NO:4 or SEQ ID NO:5 or to a transgalactosylase active fragment thereof. Still more preferably, the polypeptide has at least 95% sequence identity to SEQ ID. NO:1, SEQ ID NO:2, SEQ ID NO:3, SEQ ID NO:4 or SEQ ID NO:5 or to a transgalactosylase active fragment thereof. In yet more preferred embodiments, the polypeptide has at least 99% sequence identity to SEQ ID. NO:1, SEQ ID NO:2, SEQ ID NO:3, SEQ ID NO:4 or SEQ ID NO:5 or to a transgalactosylase active fragment thereof. Still more preferably, the polypeptide is a sequence according to SEQ ID. NO:1, SEQ ID NO:2, SEQ ID NO:3, SEQ ID NO:4 or SEQ ID NO:5 or to a transgalactosylase active fragment thereof. In yet more preferred embodiments, the polypeptide is a sequence according to SEQ ID. NO:1 or to a transgalactosylase active fragment thereof. In the most preferred embodiments, the polypeptide is a sequence according to SEQ ID. NO:1.

Preferably, the deactivation of the transgalactosylating enzyme is by heat treatment. More preferably, heat treatment is from about 70° C. to 95° C. and for between about 5 minutes to 30 minutes. Still more preferably, the heat treatment is at about 95° C. for 5 to 30 minutes. In other preferred embodiments, the heat treatment is from about 135° C. to about 150° C. for about 2 seconds to about 15 seconds.

Preferably, the lactase is a K. lactis lactase. More preferably K. lactic lactase is a polypeptide having at least 80% sequence identity to SEQ ID. NO. 6 or to a lactase active fragment thereof. Still more preferably, the K. lactis lactase comprises a polypeptide having at least 90% sequence identity to SEQ ID. NO. 6 or to a lactase active fragment thereof. In yet more preferred embodiments, the K. lactis lactase comprises a polypeptide having at least 95% sequence identity to SEQ ID. NO. 6 or to a lactase active fragment thereof. Yet more preferably, the K. lactis lactase comprises a polypeptide having at least 99% sequence identity to SEQ ID. NO. 6 or to a lactase active fragment thereof. In yet more preferred embodiments, the K. lactis lactase is a polypeptide according to SEQ ID. NO. 6 or to a lactase active fragment thereof. In the most preferred embodiments, the K. lactis lactase comprises a polypeptide according to SEQ ID. NO. 6.

Preferably, less than 20% of the GOS fiber is hydrolyzed by the lactase during the step of contacting the milk-based substrate having GOS fiber with said lactase. Still more preferably, less than about 15% of the GOS fiber is hydrolyzed. Yet more preferably, less than about 10% of the GOS fiber is hydrolyzed. In the most preferred embodiments, less than about 5% of the GOS fiber is hydrolyzed,

Preferably, the deactivation of the lactase enzyme comprises heat treatment. More preferably, the heat treatment is at about 135° C. to about 150° C. for about 2 to about 15 seconds, about 85° C. to about 115° C. for about 0.5 to about 9 seconds or at about 70° C. to about 85° C. for about 15 seconds to about 30 seconds.

Preferably, the deactivating step of the lactase is reduction of the pH of the milk-based substrate having GOS fiber and the lactase. Preferably, reduction of the pH is accomplished by adding yogurt or cheese cultures to the milk-based substrate.

Preferably, the low lactose milk-based product having GOS fiber is yoghurt, ice cream, UHT milk, flavored milk product, concentrated/condensed milk product, milk-based powder, or cheese. Preferably, the GOS fiber in the low lactose milk-based product is stable having a variance of less than about 10% within 28 days. Preferably, the low lactose milk-based product having GOS fiber contains more than about 1.5% (w/w) GOS fiber. More preferably, the low lactose milk-based product having GOS fiber contains more than about 3.2% (w/w) GOS fiber. Still more preferably, the low lactose milk-based product having GOS fiber contains more than about 4% (w/w) GOS fiber. In yet more preferred embodiments, the low lactose milk-based product having GOS fiber contains more than about 7% (w/w) GOS fiber. Still more preferably, the low lactose milk-based product having GOS fiber contains more than about 14% (w/w) GOS fiber. In yet more preferred embodiments, the low lactose milk-based product having GOS fiber contains more than about 30% (w/w) GOS fiber.

In yet another preferred embodiment of the present invention, the low lactose milk-based product having GOS fiber contains more than 1.5 g GOS fiber per 100 kcal and below 0.1% lactose or below 0.01% lactose.

In still other preferred embodiments, the lactose in the milk-based substrate has been reduced more than about 50%, more than about 97%, more than about 98%, more than about 99% or more than about 99.7%.

In other preferred embodiments of the present invention, the method further has the steps of dehydrating the low lactose milk-based product to provide a powder and dissolving the powder in water.

The present disclosure is described in further detail in the following examples, which are not in any way intended to limit the scope of the disclosure as claimed. The attached figures are meant to be considered as integral parts of the specification and description of the disclosure. The following examples are offered to illustrate, but not to limit the claimed disclosure.

### EXAMPLES

### Example 1: Enzyme Sequences

The amino acid sequence of the mature truncated form of B-galactosidase from Bifidobacterium bifidum, BIF917, is set forth as SEQ ID NO:1:

VEDATRSDSTTQMSSTPEVVYSSAVDSKQNRTSDFDANWKFMLSDSVQAQ
DPAFDDSAWQQVDLPHDYSITQKYSQSNEAESAYLPGGTGWYRKSFTIDR
DLAGKRIAINFDGVYMNATVWFNGVKLGTHPYGYSPFSFDLTGNAKFGGE
NTIVVKVENRLPSSRWYSGSGIYRDVTLTVTDGVHVGNNGVAIKTPSLAT
QNGGDVTMNLTTKVANDTEAAANITLKQTVFPKGGKTDAAIGTVTTASKS
IAAGASADVTSTITAASPKLWSIKNPNLYTVRTEVLNGGKVLDTYDTEYG
FRWTGFDATSGFSLNGEKVKLKGVSMHHDQGSLGAVANRRAIERQVEILQ
KMGVNSIRTTHNPAAKALIDVCNEKGVLVVEEVFDMWNRSKNGNTEDYGK
WFGQAIAGDNAVLGGDKDETWAKFDLTSTINRDRNAPSVIMWSLGNEMME
GISGSVSGFPATSAKLVAWTKAADSTRPMTYGDNKIKANWNESNTMGDNL
TANGGVVGTNYSDGANYDKIRTTHPSWAIYGSETASAINSRGIYNRTTGG
AQSSDKQLTSYDNSAVGWGAVASSAWYDVVQRDFVAGTYVWTGFDYLGEP
TPWNGTGSGAVGSWPSPKNSYFGIVDTAGFPKDTYYFYQSQWNDDVHTLH
ILPAWNENVVAKGSGNNVPVVVYTDAAKVKLYFTPKGSTEKRLIGEKSFT
KKTTAAGYTYQVYEGSDKDSTAHKNMYLTWNVPWAEGTISAEAYDENNRL
IPEGSTEGNASVTTTGKAAKLKADADRKTITADGKDLSYIEVDVTDANGH
IVPDAANRVTFDVKGAGKLVGVDNGSSPDHDSYQADNRKAFSGKVLAIVQ
STKEAGEITVTAKADGLQSSTVKIATTAVPGTSTEKT

The amino acid sequence of the mature truncated form of B-galactosidase from Bifidobacterium bifidum, BIF995, is set forth as SEQ ID NO:2:

VEDATRSDSTTQMSSTPEVVYSSAVDSKQNRTSDFDANWKFMLSDSVQAQ
DPAFDDSAWQQVDLPHDYSITQKYSQSNEAESAYLPGGTGWYRKSFTIDR
DLAGKRIAINFDGVYMNATVWFNGVKLGTHPYGYSPFSFDLTGNAKFGGE
NTIVVKVENRLPSSRWYSGSGIYRDVTLTVTDGVHVGNNGVAIKTPSLAT
QNGGDVTMNLTTKVANDTEAAANITLKQTVFPKGGKTDAAIGTVTTASKS
IAAGASADVTSTITAASPKLWSIKNPNLYTVRTEVLNGGKVLDTYDTEYG
FRWTGFDATSGFSLNGEKVKLKGVSMHHDQGSLGAVANRRAIERQVEILQ
KMGVNSIRTTHNPAAKALIDVCNEKGVLVVEEVFDMWNRSKNGNTEDYGK
WFGQAIAGDNAVLGGDKDETWAKFDLTSTINRDRNAPSVIMWSLGNEMME
GISGSVSGFPATSAKLVAWTKAADSTRPMTYGDNKIKANWNESNTMGDNL
TANGGVVGTNYSDGANYDKIRTTHPSWAIYGSETASAINSRGIYNRTTGG
AQSSDKQLTSYDNSAVGWGAVASSAWYDVVQRDFVAGTYVWTGFDYLGEP
TPWNGTGSGAVGSWPSPKNSYFGIVDTAGFPKDTYYFYQSQWNDDVHTLH
ILPAWNENVVAKGSGNNVPVVVYTDAAKVKLYFTPKGSTEKRLIGEKSFT
KKTTAAGYTYQVYEGSDKDSTAHKNMYLTWNVPWAEGTISAEAYDENNRL
IPEGSTEGNASVTTTGKAAKLKADADRKTITADGKDLSYIEVDVTDANGH
IVPDAANRVTFDVKGAGKLVGVDNGSSPDHDSYQADNRKAFSGKVLAIVQ
STKEAGEITVTAKADGLQSSTVKIATTAVPGTSTEKTVRSFYYSRNYYVK
TGNKPILPSDVEVRYSDGTSDRQNVTWDAVSDDQIAKAGSFSVAGTVAGQ
KISVRVTMIDEIGAL

The amino acid sequence of the mature truncated form of B-galactosidase from Bifidobacterium bifidum, BIF1068, is set forth as SEQ ID NO:3:

VEDATRSDSTTQMSSTPEVVYSSAVDSKQNRTSDFDANWKFMLSDSVQAQ
DPAFDDSAWQQVDLPHDYSITQKYSQSNEAESAYLPGGTGWYRKSFTIDR
DLAGKRIAINFDGVYMNATVWFNGVKLGTHPYGYSPFSFDLTGNAKFGGE
NTIVVKVENRLPSSRWYSGSGIYRDVTLTVTDGVHVGNNGVAIKTPSLAT
QNGGDVTMNLTTKVANDTEAAANITLKQTVFPKGGKTDAAIGTVTTASKS
IAAGASADVTSTITAASPKLWSIKNPNLYTVRTEVLNGGKVLDTYDTEYG
FRWTGFDATSGFSLNGEKVKLKGVSMHHDQGSLGAVANRRAIERQVEILQ
KMGVNSIRTTHNPAAKALIDVCNEKGVLVVEEVFDMWNRSKNGNTEDYGK
WFGQAIAGDNAVLGGDKDETWAKFDLTSTINRDRNAPSVIMWSLGNEMME
GISGSVSGFPATSAKLVAWTKAADSTRPMTYGDNKIKANWNESNTMGDNL
TANGGVVGTNYSDGANYDKIRTTHPSWAIYGSETASAINSRGIYNRTTGG
AQSSDKQLTSYDNSAVGWGAVASSAWYDVVQRDFVAGTYVWTGFDYLGEP
TPWNGTGSGAVGSWPSPKNSYFGIVDTAGFPKDTYYFYQSQWNDDVHTLH
ILPAWNENVVAKGSGNNVPVVVYTDAAKVKLYFTPKGSTEKRLIGEKSFT
KKTTAAGYTYQVYEGSDKDSTAHKNMYLTWNVPWAEGTISAEAYDENNRL
IPEGSTEGNASVTTTGKAAKLKADADRKTITADGKDLSYIEVDVTDANGH
IVPDAANRVTFDVKGAGKLVGVDNGSSPDHDSYQADNRKAFSGKVLAIVQ
STKEAGEITVTAKADGLQSSTVKIATTAVPGTSTEKTVRSFYYSRNYYVK
TGNKPILPSDVEVRYSDGTSDRQNVTWDAVSDDQIAKAGSFSVAGTVAGQ
KISVRVTMIDEIGALLNYSASTPVGTPAVLPGSRPAVLPDGTVTSANFAV
HWTKPADTVYNTAGTVKVPGTATVFGKEFKVTATIRVQ

The amino acid sequence of the mature truncated form of B-galactosidase from Bifidobacterium bifidum, BIF1172, is set forth as SEQ ID NO:4:

VEDATRSDSTTQMSSTPEVVYSSAVDSKQNRTSDFDANWKFMLSDSVQAQD
PAFDDSAWQQVDLPHDYSITQKYSQSNEAESAYLPGGTGWYRKSFTIDRDL
AGKRIAINFDGVYMNATVWFNGVKLGTHPYGYSPFSFDLTGNAKFGGENTI
VVKVENRLPSSRWYSGSGIYRDVTLTVTDGVHVGNNGVAIKTPSLATQNGG
DVTMNLTTKVANDTEAAANITLKQTVFPKGGKTDAAIGTVTTASKSIAAGA
SADVTSTITAASPKLWSIKNPNLYTVRTEVLNGGKVLDTYDTEYGFRWTGF
DATSGFSLNGEKVKLKGVSMHHDQGSLGAVANRRAIERQVEILQKMGVNSI
TTTHNPAAKALIDVCNEKGVLVVEEVFDMWNRSKNGNTEDYGKWFGQAIAG
DNAVLGGDKDETWAKFDLTSTINRDRNAPSVIMWSLGNEMMEGISGSVSGF
PATSAKLVAWTKAADSTRPMTYGDNKIKANWNESNTMGDNLTANGGVVGTN
YSDGANYDKIRTTHPSWAIYGSETASAINSRGIYNRTTGGAQSSDKQLTSY
DNSAVGWGAVASSAWYDVVQRDFVAGTYVWTGFDYLGEPTPWNGTGSGAVG
SWPSPKNSYFGIVDTAGFPKDTYYFYQSQWNDDVHTLHILPAWNENVVAKG
SGNNVPVVVYTDAAKVKLYFTPKGSTEKRLIGEKSFTKKTTAAGYTYQVYE
GSDKDSTAHKNMYLTWNVPWAEGTISAEAYDENNRLIPEGSTEGNASVTTT
GKAAKLKADADRKTITADGKDLSYIEVDVTDANGHIVPDAANRVTFDVKGA
GKLVGVDNGSSPDHDSYQADNRKAFSGKVLAIVQSTKEAGEITVTAKADGL
QSSTVKIATTAVPGTSTEKTVRSFYYSRNYYVKTGNKPILPSDVEVRYSDG
TSDRQNVTWDAVSDDQIAKAGSFSVAGTVAGQKISVRVTMIDEIGALLNYS
ASTPVGTPAVLPGSRPAVLPDGTVTSANFAVHWTKPADTVYNTAGTVKVPG
TATVFGKEFKVTATIRVQRSQVTIGSSVSGNALRLTQNIPADKQSDTLDAI
KDGSTTVDANTGGGANPSAWTNWAYSKAGHNTAEITFEYATEQQLGQIVMY
FFRDSNAVRFPDAGKTKIQI

The amino acid sequence of the mature truncated form of B-galactosidase from Bifidobacterium bifidum, BIF1241, is set forth as SEQ ID NO:5:

VEDATRSDSTTQMSSTPEVVYSSAVDSKQNRTSDFDANWKFMLSDSVQAQD
PAFDDSAWQQVDLPHDYSITQKYSQSNEAESAYLPGGTGWYRKSFTIDRDL
AGKRIAINFDGVYMNATVWFNGVKLGTHPYGYSPFSFDLTGNAKFGGENTI
VVKVENRLPSSRWYSGSGIYRDVTLTVTDGVHVGNNGVAIKTPSLATQNGG
DVTMNLTTKVANDTEAAANITLKQTVFPKGGKTDAAIGTVTTASKSIAAGA
SADVTSTITAASPKLWSIKNPNLYTVRTEVLNGGKVLDTYDTEYGFRWTGF
DATSGFSLNGEKVKLKGVSMHHDQGSLGAVANRRAIERQVEILQKMGVNSI
RTTHNPAAKALIDVCNEKGVLVVEEVFDMWNRSKNGNTEDYGKWFGQAIAG
DNAVLGGDKDETWAKFDLTSTINRDRNAPSVIMWSLGNEMMEGISGSVSGF
PATSAKLVAWTKAADSTRPMTYGDNKIKANWNESNTMGDNLTANGGVVGTN
YSDGANYDKIRTTHPSWAIYGSETASAINSRGIYNRTTGGAQSSDKQLTSY
DNSAVGWGAVASSAWYDVVQRDFVAGTYVWTGFDYLGEPTPWNGTGSGAVG
SWPSPKNSYFGIVDTAGFPKDTYYFYQSQWNDDVHTLHILPAWNENVVAKG
SGNNVPVVVYTDAAKVKLYFTPKGSTEKRLIGEKSFTKKTTAAGYTYQVYE
GSDKDSTAHKNMYLTWNVPWAEGTISAEAYDENNRLIPEGSTEGNASVTTT
GKAAKLKADADRKTITADGKDLSYIEVDVTDANGHIVPDAANRVTFDVKGA
GKLVGVDNGSSPDHDSYQADNRKAFSGKVLAIVQSTKEAGEITVTAKADGL
QSSTVKIATTAVPGTSTEKTVRSFYYSRNYYVKTGNKPILPSDVEVRYSDG
TSDRQNVTWDAVSDDQIAKAGSFSVAGTVAGQKISVRVTMIDEIGALLNYS
ASTPVGTPAVLPGSRPAVLPDGTVTSANFAVHWTKPADTVYNTAGTVKVPG
TATVFGKEFKVTATIRVQRSQVTIGSSVSGNALRLTQNIPADKQSDTLDAI
KDGSTTVDANTGGGANPSAWTNWAYSKAGHNTAEITFEYATEQQLGQIVMY
FFRDSNAVRFPDAGKTKIQISADGKNWTDLAATETIAAQESSDRVKPYTYD
FAPVGATFVKVTVTNADTTTPSGVVCAGLTEIELKTAT

The amino acid sequence of the mature form of β-galactosidase from Kluyveromyces lactis, KLac, is set forth as SEQ ID NO:6:

MSCLIPENLRNPKKVHENRLPTRAYYYDQDIFESLNGPWAFALFDAPLDAP
DAKNLDWETAKKWSTISVPSHWELQEDWKYGKPIYTNVQYPIPIDIPNPPT
VNPTGVYARTFELDSKSIESFEHRLRFEGVDNCYELYVNGQYVGFNKGSRN
GAEFDIQKYVSEGENLVVVKVFKWSDSTYIEDQDQWWLSGIYRDVSLLKLP
KKAHIEDVRVTTTFVDSQYQDAELSVKVDVQGSSYDHINFTLYEPEDGSKV
YDASSLLNEENGNTTFSTKEFISFSTKKNEETAFKINVKAPEHWTAENPTL
YKYQLDLIGSDGSVIQSIKHHVGFRQVELKDGNITVNGKDILFRGVNRHDH
HPRFGRAVPLDFVVRDLILMKKFNINAVRNSHYPNHPKVYDLFDKLGFWVI
DEADLETHGVQEPFNRHTNLEAEYPDTKNKLYDVNAHYLSDNPEYEVAYLD
RASQLVLRDVNHPSIIIWSLGNEACYGRNHKAMYKLIKQLDPTRLVHYEGD
LNALSADIFSFMYPTFEIMERWRKNHTDENGKFEKPLILCEYGHAMGNGPG
SLKEYQELFYKEKFYQGGFIWEWANHGIEFEDVSTADGKLHKAYAYGGDFK
EEVHDGVFIMDGLCNSEHNPTPGLVEYKKVIEPVHIKIAHGSVTITNKHDF
ITTDHLLFIDKDTGKTIDVPSLKPEESVTIPSDTTYVVAVLKDDAGVLKAG
HEIAWGQAELPLKVPDFVTETAEKAAKINDGKRYVSVESSGLHFILDKLLG
KIESLKVKGKEISSKFEGSSITFWRPPTNNDEPRDFKNWKKYNIDLMKQNI
HGVSVEKGSNGSLAVVTVNSRISPVVFYYGFETVQKYTIFANKINLNTSMK
LTGEYQPPDFPRVGYEFWLGDSYESFEWLGRGPGESYPDKKESQRFGLYDS
KDVEEFVYDYPQENGNHTDTHFLNIKFEGAGKLSIFQKEKPFNFKISDEYG
VDEAAHACDVKRYGRHYLRLDHAIHGVGSEACGPAVLDQYRLKAQDFNFEF
DLAFE

The amino acid sequence of the mature form of B-galactosidase from Lactobacillus delbrueckii bulgaricus, LBul, is set forth as SEQ ID NO:7

MSNKLVKEKRVDQADLAWLTDPEVYEVNTIPPHSDHESFQSQEELEEGKS
SLVQSLDGDWLIDYAENGQGPVNFYAEDFDDSNFKSVKVPGNLELQGFGQ
PQYVNVQYPWDGSEEIFPPQIPSKNPLASYVRYFDLDEAFWDKEVSLKFD
GAATAIYVWLNGHFVGYGEDSFTPSEFMVTKFLKKENNRLAVALYKYSSA
SWLEDQDFWRMSGLFRSVTLQAKPRLHLEDLKLTASLTDNYQKGKLEVEA
NIAYRLPNASFKLEVRDSEGDLVAEKLGPIRSEQLEFTLADLPVAAWSAE
KPNLYQVRLYLYQAGSLLEVSRQEVGFRNFELKDGIMYLNGQRIVFKGAN
RHEFDSKLGRAITEEDMIWDIKTMKRSNINAVRCSHYPNQSLFYRLCDKY
GLYVIDEANLESHGTWEKVGGHEDPSFNVPGDDQHWLGASLSRVKNMMAR
DKNHASILIWSLGNESYAGTVFAQMADYVRKADPTRVQHYEGVTHNRKFD
DATQIESRMYVPAKVIEEYLTNKPAKPFISVEYAHAMGNSVGDLAAYTAL
EKYPHYQGGFIWDWIDQGLEKDGHLLYGGDFDDRPTDYEFCGNGLVFADR
TESPKLANVKALYANLKLEVKDGQLFLKNDNLFTNSSSYYFLTSLLVDGK
LTYQSRPLTFGLEPGESGTFALPWPEVADEKGEVVYRVTAHLKEDLPWAD
EGFTVAEAEEVAQKLPEFKPEGRPDLVDSDYNLGLKGNNFQILFSKVKGW
PVSLKYAGREYLKRLPEFTFWRALTDNDRGAGYGYDLARWENAGKYARLK
DISCEVKEDSVLVKTAFTLPVALKGDLTVTYEVDGRGKIAVTADFPGAEE
AGLLPAFGLNLALPKELTDYRYYGLGPNESYPDRLEGNYLGIYQGAVKKN
FSPYLRPQETGNRSKVRWYQLFDEKGGLEFTANGADLNLSALPYSAAQIE
AADHAFELTNNYTWVRALSAQMGVGGDDSWGQKVHPEFCLDAQKARQLRL
VIQPLLLK

### Example 2: Enzymes

BIF917: A B-galactosidase preparation of BIF917 (ZYMSTAR™ GOS, DuPont, material A15017 batch 4863188124) having the amino acid sequence shown in SEQ ID NO:1,
KLac: A B-galactosidase preparation KLac (GODO YNL2, Godo Shusei Co.) having the amino acid sequence shown in SEQ ID NO:6
LBul: An experimental B-galactosidase preparation of LBul from Lactobacillus delbrueckii bulgaricus having the amino acid sequence shown in SEQ ID NO:6
NFit: A B-galactosidase preparation of NOLA Fit 5500 (Chr. Hansen) Material 350502, batch number 3339769, purchased through https://hjemmeriet.com

### Example 3: Method for Lactose Quantification

Samples were homogenized and diluted in H₂O following derivatization with p-aminobenzoic acid and sodium cyanoborohydride and injected on a RP-C(18) (reverse-phase) column for lactose quantification. Tetrabutylammonium hydrogen sulphate was used as the ion-pair reagent in the eluent system. Sugars were quantified fluorescence-detection (λ(ex) 313 nm, λ(em) 358 nm), at an Agilent 1100 HPLC system equipped with a Prontosil RP-C(18) SH column. The injection volume was 20 μL with a flow of 0.8 mL/min and isocratic elution using 10 mM sodium phosphate buffer containing 20 mM tetrabutylammonium bisulfate (pH 2.0). In between each injection, the column was washed with 50/50% v/v acetonitrile/water. Samples were prepared with L-Arabinose as internal standard and quantified according to a lactose standard curve.

### Example 4: Method for Quantification of GOS (DP3 and Above)

Quantification of Galacto-Oligosaccharides by HPLC

The standard lactose (HPLC analytical grade, Sigma Aldrich) was prepared in double distilled water (ddH2O) and filtered through 0.2 μm syringe filters. A dilution series ranging from 500 to 10000 ppm of the lactose standard was created.

Similar sample preparation of milk-base and yogurt samples was applied, however utilizing a dilution factor of 5× and 10× respectively. A milk-base sample was diluted 5×: 200 mg sample (weight noted) in 800 μL H2O, mixed thoroughly and inactivated 20 minutes in boiling water and following cooled. 50 μL Carrez reagent A (Carrez Clarification Kit, 1.10537.001, Sigma Aldrich) and 50 μL Carrez B was added to 1000 μL diluted sample to induce protein and lipid precipitation. The sample mixture was incubated 15 minutes at room temperature and 50 μL 10 mM NaOH, 1 mM EDTA was added to the sample. The sample was centrifuged at 10.000 rpm for 4 minutes and 300 μL clarified supernatant was transferred to an MTP filter plate, through 0.20 μm 96 well plate filters (centrifuged 3000 rpm in 15 minutes) before analysis (Corning filter plate, PVDF hydrophile membrane, NY, USA). All samples were analyzed duplicate and in 96 well MTP plates sealed with tape.

Instrumentation

Quantification of galacto-oligosaccharides (GOS), lactose, glucose and galactose were performed by HPLC. Analysis of samples was carried out on a Dionex Ultimate 3000 HPLC system (Thermo Fisher Scientific) equipped with a DGP-3600SD Dual-Gradient analytical pump, WPS-3000TSL thermostated autosampler, TCC-3000SD thermostated column oven, and a RI-101 refractive index detector (Shodex, JM Science). Chromeleon datasystem software (Version 6.80, DU10A Build 2826, 171948) was used for data acquisition and analysis.

Chromatographic Conditions

The samples were analyzed by HPLC using an RSO oligosaccharide column, Ag⁺ 4% crosslinked (Phenomenex, The Netherlands) equipped with an analytical guard column (Carbo-Ag⁺ neutral, AJ0-4491, Phenomenex, The Netherlands) operated at 70° C. The column was eluted with double distilled water (filtered through a regenerated cellulose membrane of 0.45 μm and purged with helium gas) at a flow rate of 0.3 ml/min.

Isocratic flow of 0.3 ml/min was maintained throughout analysis with a total run time of 45 minutes and injection volume was set to 20 μL. Samples were held at 30° C. in the thermostated autosampler compartment to ensure solubilization of all components. The eluent was monitored by means of a refractive index detector (RI-101, Shodex, JM Science) and quantification was made by the peak area relative to the peak area of lactose as described above. Peaks with a degree of three or higher (DP3+) were quantified as galactooligosaccharides (DP3, DP4, DP5 and so forth). The assumption of the same response for all DP3+ galacto-oligosaccharides components was confirmed with mass balances. Lactose including other DP2 components was quantified as DP2, glucose and galactose in a similar manner.

### Example 5: Stability of GOS Fibers after Pasteurization at 95° C. for 5 Minutes

It was shown that GOS fibers (DP3 and above) would be stable in a milk/yoghurt after pasteurization for 5 minutes at 95° C. in absent introduction of a secondary lactase post pasteurization.

Skimmed milk of 4.7% lactose (standardized at 4% protein and 0.03% fat) was enzymated with 3.2 g/L, of BIF917 enzyme for 18 hours at 5° C. converting lactose to GOS fibers (DP3 and above). The BIF917 enzyme was then inactivated by 5 min pasteurization at 95° C. of the milk.

Heating conditions for the pasteurization: Milk was preheated to 65° C. and homogenized at 200 bars over 30 seconds. It was then preheated to 80° C. for 4 seconds followed by pasteurization at 95° C. for 5 minutes. After pasteurization milk was cooled to 5° C.

The milk base was then heated to 43° C. Starter culture YO-MIX 495 (20DCU/100L) was added and fermentation was carried out until a pH of 4.6 was reached. All samples were then cooled to 5° C. and kept for 56 days of shelf life.

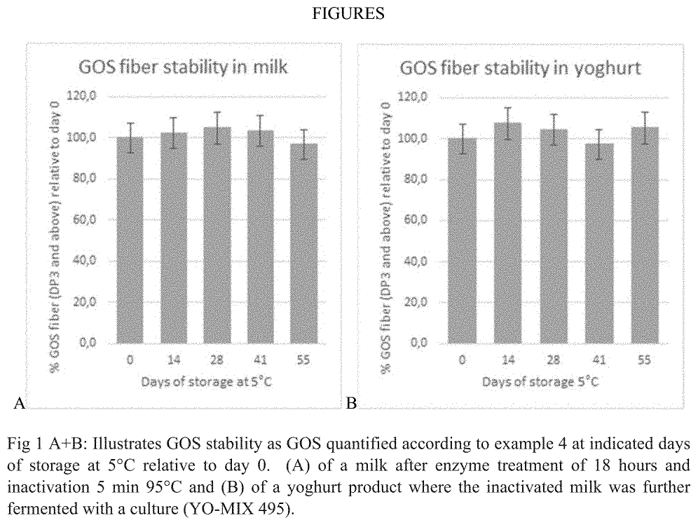

At indicated timepoints samples was quantified for GOS according to example 4. Results are shown in FIG. 1A and B.

### Example 6: Stability of GOS after UHT Pasteurization

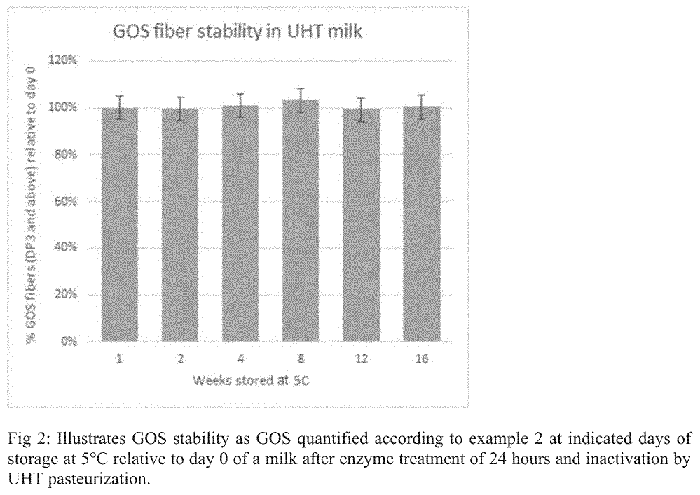

It was shown that GOS fibers (DP3 and above) would be stable in a milk after UHT pasteurization absent addition of a second lactase after pasteurization. Whole milk of 4.7% lactose was enzyme treated with 3.2 of the BIF917 and held for 24 hours at 5° C. prior to UHT pasteurization (preheated 75-80° C., 142° C. for 1-2 seconds by direct steam injection, 1500 homo psi). After pasteurization, samples were stored at 4° C. until quantification of GOS according to example 4 at timepoints as indicated in FIG. 2.

### Example 7: Residual GOS Fibers after Addition of a Second Lactase during Fermentation with a Culture

Skimmed milk of 4.7% lactose (standardized at 4% protein and 0.03% fat) was enzyme treated with 3.2 of B1F917 for 18 hours at 5° C. converting lactose to GOS fibers (DP3 and above). The BIF917 was then inactivated by 5 min pasteurization at 95° C. of the milk. Heating conditions for the pasteurization: Milk was preheated to 65° C. and homogenized at 200 bars over 30 seconds. It was then preheated to 80° C. for 4 seconds followed by pasteurization at 95° C. for 5 minutes. After pasteurization milk was cooled to 5° C.

Following, the milk-base was fermented to a yoghurt. The milk base was heated to 43° C.

Various amounts of either KLac, LBul or NFit lactase was added according to table 1 together with starter culture YO-MIX 495 (20DCU/100L, Dupont Nutrition Biosciences). Fermentation was carried out until a pH of 4.6 was reached in which enzymes (KLac and LBul) were inactivated by the decrease in ph. All samples were then cooled to 5° C. and then frozen 24 hours after end of fermentation until they were analyzed for residual lactose and GOS according to example 3 and 4. Results are shown in table 1.

TABLE 1
The residual GOS fibers (relative to sample without secondary lactase
added) and lactose reduction (relative to initial lactose concentration
of milk-base or relative to sample without secondary lactase
added) after fermentation with culture with indicated
doses of lactase in the GOS containing milk base.

| | % residual GOS | % total lactose | % lactose reduced by |
|---|---|---|---|
| | fibers | reduction | lactase |
| g/L KLac | | | |
| 0.00 | 100 | 86.17 | 0.00 |
| 0.05 | 98 | 92.13 | 43.08 |
| 0.10 | 99 | 96.17 | 72.31 |
| 0.20 | 95 | 98.94 | 92.31 |
| 0.40 | 85 | 99.85 | 98.92 |
| g/L LBul | | | |
| 0.00 | 100 | 86.17 | 0.00 |
| 0.05 | 94 | 91.28 | 36.92 |
| 0.10 | 87 | 95.53 | 67.69 |
| 0.20 | 74 | 97.45 | 81.54 |
| 0.40 | 41 | 98.74 | 90.92 |
| g/L NFit | | | |
| 0.00 | 100 | 78.72 | 0.00 |
| 0.06 | 77 | 89.36 | 50.00 |
| 0.08 | 70 | 90.85 | 92.13 |
| 0.10 | 64 | 92.13 | 63.00 |
| 0.12 | 61 | 94.04 | 72.00 |

### Example 8: Residual GOS Fibers After Addition of a Second Lactase to the GOS Enriched UHT Milk

Skim milk of 4.7% lactose was enzymated treated with 3.2 g/L BIF917 for 24 hours at 5° C. After 24 hours, the milk was UHT pasteurized (preheated 75-80° C., 142° C. for 1-2 seconds by direct steam injection, 1500 homo psi). The milk enzymated with 3.2 g/L of BIF917 was then added the second lactase being either LBul, KLac or NFit dosed according to table 2 and incubated 5° C. for 24 hours. Samples was then heat treated at 95° C. for 20 minutes prior to analyzing on HPLC. Lactose and GOS fibers were quantified according to example 3 and 4.

TABLE 2
The residual GOS fibers (relative to sample without secondary lactase
added) and lactose reduction (relative to initial lactose concentration
of milk-base or relative to sample without secondary lactase added)
after fermentation with culture with indicated doses of lactase in
the GOS containing milk base.

| | % residual GOS | % total lactose | % lactose reduced by |
|---|---|---|---|
| | fibers | reduction | lactase |
| g/L KLac | | | |
| 0.00 | 100 | 78.28 | 0.00 |
| 0.35 | 99 | 99.13 | 95.98 |
| 0.50 | 94 | 99.83 | 99.21 |
| 1.00 | 88 | 99.94 | 99.71 |
| g/L LBul | | | |
| 0.00 | 100 | 78.28 | 0.00 |
| 0.35 | 78 | 97.72 | 89.52 |
| 0.50 | 66 | 98.36 | 92.46 |
| 1.00 | 33 | 98.79 | 94.42 |
| g/L NFit | | | |
| 0.00 | 100 | 78.28 | 0.00 |
| 0.35 | 66 | 91.38 | 60.33 |
| 0.50 | 57 | 94.62 | 75.22 |
| 1.00 | 38 | 97.64 | 89.13 |

Although the foregoing invention has been described in some detail by way of illustration and example, for purposes of clarity of understanding, certain changes and modifications can be practiced within the scope of the appended claims. In addition, each reference provided herein is incorporated by reference in its entirety for all purposes to the same extent as if each reference was individually incorporated by reference. To the extent the content of any citation, including website or accession number may change with time, the version in effect at the filing date of this application is meant. Unless otherwise apparent from the context any step, element, aspect, feature of embodiment can be used in combination with any other.

1. A method for preparing a low lactose milk-based product having GOS fiber, the method comprising the steps of providing a milk-based substrate comprising lactose;
treating said milk-based substrate with a transgalactosylating enzyme to provide GOS fiber and remaining lactose;
deactivating the transgalactosylating enzyme;
contacting the milk-based substrate having GOS fiber with a lactase to degrade the remaining lactose to provide the low lactose milk-based product having GOS fiber; and
deactivating the lactase.

2. The method of claim 1 wherein the milk-based substrate has a lactose concentration of between 1-60% (w/w); or 2-50% (w/w), or 3-40% (w/w); or 4-30% (w/w).

3. The method of claim 1 wherein the transgalactosylating enzyme is a truncated β-galactosidase from Bifidobacterium bifidum.

4. The method of claim 3 wherein the truncated β-galactosidase from Bifidobacterium bifidum is truncated on the C-terminus.

5. The method of claim 4 wherein the truncated β-galactosidase from Bifidobacterium bifidum comprises a polypeptide having at least 70% sequence identity to SEQ ID. NO:1, SEQ ID NO:2, SEQ ID NO:3, SEQ ID NO:4 or SEQ ID NO:5 or to a transgalactosylase active fragment thereof.

6. The method of claim 5 wherein the polypeptide has at least 80% sequence identity to SEQ ID. NO:1, SEQ ID NO:2, SEQ ID NO:3, SEQ ID NO:4 or SEQ ID NO:5 or to a transgalactosylase active fragment thereof.

7. The method of claim 6 wherein the polypeptide has at least 90% sequence identity to SEQ ID. NO:1, SEQ ID NO:2, SEQ ID NO:3, SEQ ID NO:4 or SEQ ID NO:5 or to a transgalactosylase active fragment thereof.

8. The method of claim 7 wherein the polypeptide has at least 95% sequence identity to SEQ ID. NO:1, SEQ ID NO:2, SEQ ID NO:3, SEQ ID NO:4 or SEQ ID NO:5 or to a transgalactosylase active fragment thereof.

9. The method of claim 8 wherein the polypeptide has at least 99% sequence identity to SEQ ID. NO:1, SEQ ID NO:2, SEQ ID NO:3, SEQ ID NO:4 or SEQ ID NO:5 or to a transgalactosylase active fragment thereof.

10. The method of claim 9 wherein the polypeptide comprises a sequence according to SEQ ID. NO:1, SEQ ID NO:2, SEQ ID NO:3, SEQ ID NO:4 or SEQ ID NO:5 or to a transgalactosylase active fragment thereof.

11. The method of claim 10 wherein the polypeptide comprises a sequence according to SEQ ID. NO:1 or to a transgalactosylase active fragment thereof.

12. The method of claim 11 wherein the polypeptide comprises a sequence according to SEQ ID. NO:1.

13. The method of claim 1 wherein the deactivation of the transgalactosylating enzyme comprises heat treatment.

14. The method of claim 13 wherein heat treatmentis from about 70° C. to 95° C. and for between about 5 minutes to 30 minutes.

15. The method of claim 14 wherein the heat treatment is at about 95° C. for 5 to 30 minutes.

16. The method of claim 13 wherein the heat treatment is from about 135° C. to about 150° C. for about 2 seconds to about 15 seconds.

17. The method of claim 1 wherein the lactase is a K. lactis lactase.

18. The method according to claim 17 wherein the K. lactis lactase comprises a polypeptide having at least 80% sequence identity to SEQ ID. NO. 6 or to a lactase active fragment thereof.

19. The method according to claim 18 wherein the K. lactis lactase comprises a polypeptide having at least 90% sequence identity to SEQ ID. NO. 6 or to a lactase active fragment thereof.

20. The method according to claim 19 wherein the K. lactis lactase comprises a polypeptide having at least 95% sequence identity to SEQ ID. NO. 6 or to a lactase active fragment thereof.

21. The method according to claim 20 wherein the K. lactis lactase comprises a polypeptide having at least 99% sequence identity to SEQ ID. NO. 6 or to a lactase active fragment thereof.

22. The method according to claim 21 wherein the K. lactis lactase comprises a polypeptide according to SEQ ID. NO. 6 or to a lactase active fragment thereof.

23. The method according to claim 22 wherein the K. lactis lactase comprises a polypeptide according to SEQ ID. NO. 6.

24. The method of of claim 1 wherein less than 20% of the GOS fiber is hydrolyzed by the lactase during the step of contacting the milk-based substrate having GOS fiber with said lactase.

25. The method of claim 24 wherein less than about 15% of the GOS fiber is hydrolyzed.

26. The method of claim 25 wherein less than about 10% of the GOS fiber is hydrolyzed.

27. The method of claim 26 wherein less than about 5% of the GOS fiber is hydrolyzed.

28. The method of claim 1 wherein the deactivation of the lactase enzyme comprises heat treatment.

29. The method according to 28 wherein the heat treatment is at about 135° C. to about 150° C. for about 2 to about 15 seconds, about 85° C. to about 115° C. for about 0.5 to about 9 seconds or at about 70° C. to about 85° C. for about 15 seconds to about 30 seconds.

30. The method claim 1 wherein the deactivating step of the lactase comprises reduction of the pH of the milk-based substrate having GOS fiber and the lactase.

31. The method according to claim 30 wherein reduction of the pH is accomplished by adding yogurt or cheese cultures to the milk-based substrate.

32. The method of claim 1 wherein the low lactose milk-based product having GOS fiber is yoghurt, ice cream, UHT milk, flavored milk product, concentrated/condensed milk product, milk-based powder, or cheese.

34. The method of claim 1 wherein the GOS fiber in the low lactose milk-based product is stable having a variance of less than about 10% within 28 days.

35. The method of claim 1 wherein the low lactose milk-based product having GOS fiber contains more than about 1.5% (w/w) GOS fiber.

36. The method according to claim 35 wherein the low lactose milk-based product having GOS fiber contains more than about 3.2% (w/w) GOS fiber.

37. The method according to claim 36 wherein the low lactose milk-based product having GOS fiber contains more than about 4% (w/w) GOS fiber.

38. The method according to claim 37 wherein the low lactose milk-based product having GOS fiber contains more than about 7% (w/w) GOS fiber.

39. The method according to claim 38 wherein the low lactose milk-based product having GOS fiber contains more than about 14% (w/w) GOS fiber.

40. The method according to claim 39 wherein the low lactose milk-based product having GOS fiber contains more than about 30% (w/w) GOS fiber.

42. The method of claim 1 wherein the low lactose milk-based product having GOS fiber contains more than 1.5 g GOS fiber per 100 kcal and below 0.1% lactose or below 0.01% lactose.

43. The method of claim 1 wherein the lactose in the milk-based substrate has been reduced more than about 50%, more than about 97%, more than about 98%, more than about 99% or more than about 99.7%.

44. The method of claim 1 further comprising the steps of dehydrating the low lactose milk-based product to provide a powder and dissolving the powder in water.
